(19) United States
(12) Patent Application Publication
(10) Pub. No.: US 2014/0086794 A1
(43) Pub. Date: Mar. 27, 2014

(54) PREVENTION METHOD AND PREVENTIVE SOLIDIFIED SUBSTANCE, METHOD FOR APPLYING PREVENTIVE SOLIDIFIED SUBSTANCE, BASE MATERIAL AND MATERIAL USED IN APPLICATION OF PREVENTIVE SOLIDIFIED SUBSTANCE, AND METHOD FOR PRODUCING PREVENTING SOLIDIFIED SUBSTANCE

(71) Applicants: Junzou Maruyama, Amagasaki-shi (JP); Tetsuya Matsumoto, Amagasaki-shi (JP); Shigeru Fukumoto, Kagoshima-shi (JP); Junichi Nakamizo, Kagoshima-shi (JP); Yasuto Hirakawa, Kumamoto-shi (JP)

(72) Inventors: Junzou Maruyama, Amagasaki-shi (JP); Tetsuya Matsumoto, Amagasaki-shi (JP); Shigeru Fukumoto, Kagoshima-shi (JP); Junichi Nakamizo, Kagoshima-shi (JP); Yasuto Hirakawa, Kumamoto-shi (JP)

(21) Appl. No.: 14/002,848

(22) PCT Filed: Feb. 29, 2012
(86) PCT No.: PCT/JP2012/055065

§ 371 Date: Nov. 19, 2013

(30) Foreign Application Priority Data
Mar. 4, 2011 (JP) 2011-047603

(51) Int. Cl. A61L 2/232; A01N 25/02

(52) U.S. Cl. CPC A61L 2/232; A01N 25/02
USPC 422/28; 424/405

(57) ABSTRACT

Provided is an epidemic prevention method including providing a mixed slurry comprising an emulsion of a non-alkaline-thickening resin which is free from a coagulation-attributable viscosity increase even when mixed with an alkaline substance and an alkaline substance of which a diluted solution has pH 10.0 or more, and coating, drying, and solidifying the mixed slurry to internally disperse and embed the alkaline substance in the form of solid particles and simultaneously to form a solidified matter provided with microcracks communicated with the solid particles of the internal alkaline substance, so that a moisture which adheres to a surface of the solidified matter permeates through the microcracks and dissolves the solid particles of the internal alkaline substance to cause alkalinity of pH 10.0 or more to be exhibited on the surface of the solidified matter, thereby inactivating microbes and viruses on the surface of the solidified matter.

### TECHNICAL FIELD

Embodiments described herein relate generally to an epidemic prevention method for inactivating viruses and microbes contacting a surface and an epidemic prevention solidified matter to be used for the method.

### BACKGROUND ART

In the livestock industry, particularly in the field of rearing domestic poultry such as chickens and livestock such as cows and pigs, highly pathogenic avian influenza (HPAI) and porcine and bovine viruses such as a circovirus and a foot-and-mouth disease virus have raged to inflict severe and huge damages. The avian influenza and porcine and bovine viruses are designated as the notifiable infectious diseases in Act on Domestic Animal Infectious Disease Control and, therefore, it is necessary to take the appropriate measure against them and to prevent them from invading into a livestock barn and a poultry house and infecting the livestock and the poultry.

As a method for preventing invasion of pathogenic viruses and bacteria and for protecting livestock and poultry from damages of the influenza and foot-and-mouth disease by killing or inactivating pathogens even when the invasion occurs, a countermeasure of spraying a lime hydrate powder inside a premise of a poultry house or a bovine/porcine barn or at entrances of the premise has generally been taken. The countermeasure has the effects of epidemic prevention and disinfection against the avian influenza and bovine and porcine influenza viruses, and the lime hydrate exhibits strong alkalinity when dissolved into water to kill or inactivate the avian influenza and bovine and porcine influenza viruses.

As to lime hydrate, JP 2003-49122 A discloses one example of an aqueous coating material containing lime hydrate.

### SUMMARY OF INVENTION

### Technical Problem

However, the aqueous coating material disclosed in JP 2003-49122 A relates to the technology of preventing efflorescence, not to the epidemic prevention method.

In view of the above-described current situation, an object of the present invention is to provide an epidemic prevention method which contains an alkaline substance and enables inactivation of microbes and viruses contacting a surface and an epidemic prevention solidified matter used for the epidemic prevention method.

### Solution to Problem

The epidemic prevention method according to the present invention is characterized by:

using as a matrix a mixed slurry comprising an emulsion of a non-alkaline-thickening resin which is free from a coagulation-attributable viscosity increase even when mixed with an alkaline substance and an alkaline substance of which a diluted solution has pH 10.0 or more and being obtainable by mixing and stirring the emulsion and the alkaline substance;

coating, drying, and solidifying the matrix on a surface of an object to form on the surface of the object a solidified matter in which the alkaline substance is internally dispersed and embedded in the form of solid particles and microcracks communicated with the solid particles of the internal alkaline substance are formed;

allowing a moisture adhered to a surface of the solidified matter to permeate through the microcracks thereby to dissolve the solid particles of the internal alkaline substance; and

inactivating a microbe or a virus on the surface of the solidified matter by exhibiting pH 10.0 or more on the surface of the solidified matter.

According to the epidemic prevention method of the present invention, by allowing water to adhere to the surface of the solidified matter during raining or sprinkling or by allowing natural dew condensation water to adhere to the surface of the solidified matter, the water adhered to the surface of the solidified matter permeates through the microcracks to dissolve the solid particles of the internal alkaline substance and causes the alkalinity of pH 10.0 or more to be exhibited on the surface of the solidified matter. Therefore, pathogenic viruses such as an avian influenza virus and a porcine influenza virus adhered to the surface of the solidified matter are inactivated, and thus an antiviral effect is attained.

In the epidemic prevention method of the present invention, since the alkaline substance which is dispersed and embedded in the form of solid particles shielded from the air inside the solidified matter and the surface of the solidified matter are communicated with each other via the microcracks, the internal alkaline substance gradually exudes out onto the solidified matter surface through the microcracks. Meanwhile, since the alkaline substance other than the exuded out one, which remains dispersed and embedded inside the solidified matter, is still in the state of being shielded from the air, the alkaline substance is not neutralized with a carbonate gas in the air and, therefore, is not changed into a neutralized product such as calcium carbonate even when a hydroxide such as lime hydrate is used. Thus, the antibacterial and antiviral effect is exhibited for a long time. The alkaline substance may be fine particles or secondary particles formed by aggregation of the fine particles.

Emulsified resins in general have the property of being increased in viscosity by coagulation when brought into contact with an alkaline substance such as strong alkali. Accordingly, there is a problem that the emulsified resin becomes an ice cream-like form to lose fluidity when it is mixed with an alkaline substance powder such as strong alkali and makes it difficult to form a solidified matter in the form of a coating film. In contrast, the resin for forming the solidified matter according to the present invention is the non-alkaline-thickening resin. Therefore, the resin is not increased in viscosity even when the alkaline substance particle such as strong alkali is added thereto with stirring and is suitably coated on a surface of an object to be prevented from epidemic.

A solidified matter to be used for the epidemic prevention method according to the present invention is characterized in that:

a mixed slurry comprising an emulsion of a non-alkaline-thickening resin which is free from a coagulation-attributable viscosity increase even when mixed with an alkaline substance and an alkaline substance of which a diluted solution has pH 10.0 or more and being obtainable by mixing and stirring the emulsion and the alkaline substance is comprised as a matrix; and

the alkaline substance is internally dispersed and embedded in the form of solid particles and, simultaneously, the solid particles of the internal alkaline substance and an outer surface are communicated with each other via microcracks, the solid particles and the microcracks being formed by coating, drying, and solidifying the matrix on a surface of an object.

The solidified matter used for the epidemic prevention method according to the present invention has excellent weatherability, and the internal alkaline substance is not fallen off from the solidified matter surface even when the solidified matter is exposed to strong wind and heavy rain. Therefore, the antibacterial and antiviral effect is exhibited for a long time.

A method for forming the solidified matter used for the epidemic prevention method according to the present invention is characterized by:

preparing a mixed slurry as a matrix by mixing and stirring an emulsion of a non-alkaline-thickening resin which is free from a coagulation-attributable viscosity increase even when mixed with an alkaline substance, an alkaline substance of which a diluted solution has pH 10.0 or more, and concentration adjustment water; and

coating, drying, and solidifying the mixed slurry on a surface of an object to form on the surface of the object a solidified matter in which the alkaline substance is dispersed and embedded in the form of solid particles and microcracks which provide communication between the solid particles of the internal alkaline substance and an outer surface are formed.

According to the formation method, since the mixed slurry only has to be conveyed into and coated on the formation site where the antibacterial and antiviral effect is desired, the excellent workability is attained.

A matrix used for the formation of the solidified matter used for the epidemic prevention method according to the present invention is firstly characterized by

comprising an emulsion of a non-alkaline-thickening resin which is free from a coagulation-attributable viscosity increase even when mixed with an alkaline substance, an alkaline substance of which a diluted solution has pH 10.0 or more, and concentration adjustment water and being prepared as a mixed slurry obtainable by mixing and stirring the emulsion, the alkaline substance, and the concentration adjustment water.

The matrix used for the formation of the solidified matter used for the epidemic prevention method according to the present invention is secondly characterized in that

the alkaline substance comprises at least one or a mixture of plural kinds selected from oxides of quicklime and magnesium oxide, lime hydrate, magnesium hydroxide, hydroxides of sodium hydroxide and potassium hydroxide, and alkali salts of sodium carbonate, potassium carbonate, sodium silicate, potassium silicate, sodium phosphate, potassium phosphate, sodium acetate, and potassium acetate.

The matrix used for the formation of the solidified matter used for the epidemic prevention method according to the present invention is thirdly characterized in that

the matrix is the mixed slurry comprising 15 to 75 parts by weight of the emulsion containing 5 to 45 parts by weight of the non-alkali-thickening resin, 10 to 50 parts by weight of alkaline substance, and 5 to 40 parts by weight of concentration adjustment water based on 100 parts by weight of the entire mixed slurry.

The matrix used for the formation of the solidified matter used for the epidemic prevention method according to the present invention is fourthly characterized in that

the non-alkali-thickening resin is at least one resin or a resin mixture of plural kinds of resins selected from an acrylic resin, a urethane resin, a fluorine resin, a silicon resin, a polyester resin, a polyethylene resin, a polystyrene resin, an epoxy resin, and a polyphenol resin.

The acrylic resin contains an acrylic acid ester or mainly an acrylic acid ester and a small amount of a urethane resin, a silicon resin, and/or an ethylene vinyl acetate resin (EVA).

The matrix used for the formation of the solidified matter used for the epidemic prevention method according to the present invention is fifthly characterized by

comprising as an auxiliary material one kind or a mixture of plural kinds selected from a coloring agent, a spreadability enhancer, an antibacterial agent, an antiviral agent, a viscosity improver, a surfactant, a dispersant, an antifoaming agent, and heat shielding particles.

The auxiliary agent further comprises 7.4 to 10 parts by weight. Titanium oxide in an amount of 7 to 10 parts by weight may further be contained. According to the embodiment, it is possible to make the coating film more wettable with water by reducing a contact angle of water droplets adhered to the coating film and to make the coating film less subject to falling off by improving spreadability of the coating film on the object. Also, in view of the fact that titanium oxide is used as a white pigment, it is possible to form a white coating film thereby to attain reflection, light shielding, and heat shielding effects against sunlight.

The matrix used for the formation of the solidified matter used for the epidemic prevention method according to the present invention is sixthly characterized by

comprising one kind or plural kinds of heat shielding particles selected from titanium oxide, perlite, ferrite, and ceramic particles as the auxiliary agent among the auxiliary agents.

A matrix adjustment material used for the formation of the solidified matter used for the epidemic prevention method according to the present invention is firstly characterized in that:

the material is prepared as a mixed slurry by mixing and stirring one kind or plural kinds of the auxiliary agents and an emulsion of a non-alkaline-thickening resin which is free from a coagulation-attributable viscosity increase even when mixed with an alkaline substance; and the thus-prepared material is mixed and stirred with a predetermined amount of concentration adjustment water and then with an alkaline substance of which a diluted solution exhibits pH of 10.0 or more to be prepared into a mixed slurry to be used as the matrix when implementing the formation method.

A matrix adjustment material used for the formation of the solidified matter used for the epidemic prevention method according to the present invention is secondly characterized in that:

the material is prepared as a cake-like solid matter by adding water to one kind or plural kinds of the alkaline substances and one kind or plural kinds of the auxiliary agents, followed by kneading; and

the thus-prepared material and an emulsion of a non-alkaline-thickening resin which is free from a coagulation-attributable viscosity increase even when mixed with an alkaline substance are mixed and stirred to be prepared into a mixed slurry to be used as the matrix when implementing the formation method.

An epidemic prevention material on which a coating film of the solidified matter used in the epidemic prevention method according to the present invention is formed is firstly characterized in that:

the material comprises, as a matrix, an emulsion of a non-alkaline-thickening resin which is free from a coagulation-attributable viscosity increase even when mixed with an alkaline substance, an alkaline substance of which a diluted solution has pH 10.0 or more, and concentration adjustment water, and the emulsion, the alkaline substance, and the concentration adjustment water being mixed and stirred to be prepared into a mixed slurry; and

the mixed slurry is coated, dried, and solidified on each of surfaces of one kind or plural kinds of grains selected from pebbles, gravel, sand, pumice, cobbles, wood chip, metal grains, concrete grains, ceramic grains, and plastic grains to form the epidemic prevention solidified matter in the form of a coating film on the surface of each of the grains.

The epidemic prevention material on which a coating film of the solidified matter used in the epidemic prevention method according to the present invention is formed is secondly characterized in that:

the material comprises, as a matrix, an emulsion of a non-alkaline-thickening resin which is free from a coagulation-attributable viscosity increase even when mixed with an alkaline substance, an alkaline substance of which a diluted solution has pH 10.0 or more, and concentration adjustment water, and the emulsion, the alkaline substance, and the concentration adjustment water being mixed and stirred to be prepared into a mixed slurry; and

the mixed slurry is coated, dried, and solidified on a surface of one kind selected from sheet materials such as nonwoven cloth, cloth, paper, a mat, a carpet, a rug, an artificial grass, and a film to form the epidemic prevention solidified matter in the form of a coating film on the surface of the sheet material.

A process for producing the solidified matter used for the epidemic prevention method according to the present invention is firstly characterized by

preparing a mixed slurry as a matrix by mixing and stirring an emulsion of a non-alkaline-thickening resin which is free from a coagulation-attributable viscosity increase even when mixed with an alkaline substance, an alkaline substance of which a diluted solution has pH 10.0 or more, and concentration adjustment water; and

coating, drying, and solidifying the mixed slurry on each of surfaces of one kind or plural kinds of grains selected from pebbles, gravel, sand, pumice, cobbles, wood chip, metal grains, concrete grains, ceramic grains, and plastic grains to form the epidemic prevention solidified matter in the form of a coating film on the surface of each of the grains. In this case, when using the grains in each of which the coating film-like solidified matter is formed on the surface, a formation method of spreading, stacking, floating, or immersing the grains in a site of epidemic prevention is performed.

The process for producing the solidified matter used for the epidemic prevention method according to the present invention is secondly characterized by

preparing a mixed slurry as a matrix by mixing and stirring an emulsion of a non-alkaline-thickening resin which is free from a coagulation-attributable viscosity increase even when mixed with an alkaline substance, an alkaline substance of which a diluted solution has pH 10.0 or more, and concentration adjustment water; and

coating, drying, and solidifying the mixed slurry on a surface of one kind selected from sheet materials such as nonwoven cloth, cloth, paper, a mat, a carpet, a rug, an artificial grass, and a film to form the epidemic prevention solidified matter in the form of a coating film on the surface of the sheet material. In this case, when using the sheet material in which the coating film-like solidified matter is formed on the surface, a formation method of spreading, attaching, or hanging the sheet material in a site where epidemic prevention is performed.

The process for producing the solidified matter used for the epidemic prevention method according to the present invention is thirdly characterized by

preparing a mixed slurry as a matrix by mixing and stirring an emulsion of a non-alkaline-thickening resin which is free from a coagulation-attributable viscosity increase even when mixed with an alkaline substance, an alkaline substance of which a diluted solution has pH 10.0 or more, and concentration adjustment water; and

casting the mixed slurry into a molding die, followed by drying and solidification to obtain a block-like or flat plate-like solidified matter in which the alkaline substance is internally dispersed and embedded in the form of solid particles and microcracks provide communication between the solid particles of the internal alkaline substance and an outer surface.

The epidemic prevention method according to the prevent invention is secondly characterized by subjecting the outer surface of the solidified matter to brushing or cleaning with high pressure water.

The avian influenza virus invades into a poultry house or livestock barn when a wild bird coming from outdoors rests on and leave its excrement on a roof material, a beam material, a rainwater guttering, or the like of the poultry house or livestock barn. Also, a pathogenic virus invades into a poultry house or livestock barn when a small animal such as a field mouse runs over and leaves its excrement on an outer wall material, a floor material, or the like. Therefore, with the implementation of the present invention, it is possible to enjoy epidemic prevention and disinfection effects against the pathogenic viruses.

The epidemic prevention method according to the prevent invention is thirdly characterized by

using as a matrix a mixed slurry comprising an emulsion of a non-alkaline-thickening resin which is free from a coagulation-attributable viscosity increase even when mixed with an alkaline substance and an alkaline substance of which a diluted solution has pH 10.0 or more and being obtainable by mixing and stirring the emulsion and the alkaline substance; coating, drying, and solidifying the matrix on a surface of an object to form on the surface of the object a solidified matter in which the alkaline substance is internally dispersed and embedded in the form of solid particles and microcracks communicated with the solid particles of the internal alkaline substance are formed; allowing a moisture adhered to a surface of the solidified matter to permeate through the microcracks thereby to dissolve the solid particles of the internal alkaline substance; and preventing a pest, a small animal, or a microscopic animal from depositing on the surface of the solidified matter and invading beyond a region where the solidified matter is formed, by exhibiting pH 10.0 or more on the surface of the solidified matter.

The process for producing the solidified matter used for the epidemic prevention method according to the present invention is fourthly characterized by:

preparing a mixed slurry as a matrix by mixing and stirring an emulsion of a non-alkaline-thickening resin which is free from a coagulation-attributable viscosity increase even when mixed with an alkaline substance, an alkaline substance of which a diluted solution has pH 10.0 or more, and concentration adjustment water; and coating, drying, and solidifying the mixed slurry on a surface of one kind of object selected from a trunk or branches of a tree which is subject to encroachment by insects, microscopic animals and plants, ticks or microbes, a timber of the tree, a livestock barn or a structure inside a house which can be a habitat of microscopic animals and plants, a structure in water on which small animals or microscopic animals and plant can be deposited, and a soil or a ground, so that a solidified matter in the form of a coating film is formed on the surface of the object.

According to the present invention, pathogenic viruses and bacteria on the surface of the solidified matter are inactivated by: coating the mixed slurry on a vehicle, a fixture, a road, a premise, a feed tank, a fence, an outer surface of a piping, a circumference, a roof of a building, a building, or a structure enclosing a region to be protected, a paved peripheral region enclosing a building or a region to be protected, or a region to be protected; drying and curing the coated region to form the solidified matter, e.g. a solidified matter in the form of a coating film, having exuding paths communicated with the alkaline substance; dissolving the alkaline substance with a moisture which adheres to the surface of the solidified matter and permeates through the exuding paths to allow pH 10 or more to be expressed on the surface of the coating film.

The circumference of building means a roof material or an outer wall material of the building, for example. Also, the mixed slurry may be coated on an inner wall material, a beam material, a column material, a rainwater guttering, or a floor material of the building. The structure enclosing building or region to be protected means a fence, for example. The paved peripheral region enclosing building or region to be protected means a peripheral road, for example.

According to the present invention, it is possible to prevent spread of pathogenic viruses and bacterial caused by depositing on cars and fixtures. Alternatively, it is possible to prevent transfer beyond a road or a premise. Alternatively, it is possible to prevent the spread which can be caused through a medium such as a premise, a feed tank, a fence, and an outer surface of piping. Alternatively, it is possible to prevent invasion into a building or an area to be protected. Alternatively, it is possible to prevent adherence and spread of microbes, mushrooms. The building includes those used by human beings as a residence or a site of activity as well as a site of breeding mammals or birds for the purpose of obtaining meat or eggs, such as a livestock barn and a poultry house, and a site of feeding mammals or birds for other purposes such as research and appreciation. Therefore, every sites of feeding animals in general are included without limitation to the poultry house or porcine barn. Further, the region to be protected may be a site of growing a plant.

According to the present invention, there will be created various applications and usages such as: as an application associated with the object to be coated, use of carpeting such as a mat and an artificial grass for epidemic prevention, use of cloth such as nonwoven cloth, a paper such as a wallpaper, or a film for epidemic prevention or heat shielding, use of the matrix of the present invention coated on trunks or branches of trees for pest control and epidemic prevention, use of the matrix of the present invention coated on an interior or an exterior of a vehicle for epidemic prevention or heat shielding, use of the matrix of the present invention coated on a picket or a net in a cultivation area of fish, oyster, seaweed, or the like or on a portion of a port facility or a ship under the sea for prevention of shell deposition and epidemic prevention, and use of the matrix of the present invention coated on a net such as an insect net and a bird net used in agriculture, livestock industry, houses and buildings, and so forth and a lawn for invasion prevention, epidemic prevention, and heat shielding; as an application idea associated with the auxiliary material, enhancement of a function of each of agents such as pest control agents including a termite control agent and a pine weevil control agent by adding the agent; as an application idea associated with the control method, a control method using a pH indicator or a pH test paper and a control method for recovering high alkalinity by performing cleaning by brushing with water, cleaning with high pressure water, or cleaning with an agent which dissolves calcium carbonate; a method which enables coating immediately after sandblast cleaning of a surface to be coated without drying and curing; as a control means of keeping a SRS coating surface such as an entrance of a premise of a business facility or a house always wet or wetting the SRS coating surface as required, always supplying water or supplying water as required by providing a sprinkler equipment on both sides or an upper part or by embedding a water dripping pipe in the SRS coating surface; and so forth. Also, by coating a snow melting agent such as potassium chloride in place of the highly alkaline substance of the present invention such as lime hydrate on a road or roof, a road which is not frozen or a roof not requiring snow removal can be realized.

### Advantageous Effects of Invention

According to the present invention, since the solidified matter is provided with the exuding paths, the alkaline substance inside the solidified matter gradually exudes out to be dissolved, thereby stably exhibiting the alkalinity of 10.0 or more. Therefore, it is possible to stably exhibit the antibacterial and antiviral properties and to inactivate pathogenic viruses and bacteria, and, furthermore, since the alkaline substance dispersed and embedded inside the solidified matter is in the state of being shielded from the air, it is not neutralized with a carbonate gas nor is deteriorated, thereby attaining the excellent effect of stably exhibiting the epidemic prevention and disinfection effects for a long time.

### BRIEF DESCRIPTION OF DRAWING

FIG. 1 is a block diagram showing a cross-section of an epidemic prevention solidified matter according to the first embodiment of the present invention.

### DESCRIPTION OF EMBODIMENTS

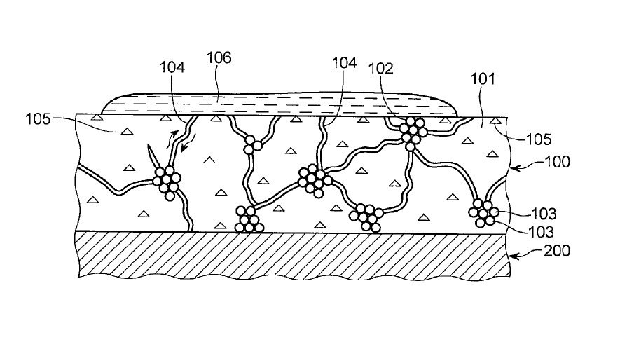

Hereinafter, the embodiments of the present invention will be described in detail with reference to the drawing. FIG. 1 is a block diagram showing a cross-section of an epidemic prevention solidified matter according to the first embodiment of the present invention, and the reference numeral 100 in the drawing denote an epidemic prevention solidified matter in the form of a coating film according to the present invention.

The epidemic prevention solidified matter 100 is a solidified matter in the form of a coating film coating a surface of a base material 200 and comprises a non-alkali-thickening acrylic resin 101, lime hydrate particles 102, and titanium oxide particles 103. The lime hydrate particles 102 comprise secondary particles each of which is a coagulation of fine particles 103, and the size of the lime hydrate particles 102 is 5 to 100 μm, chiefly 10 to 50 μm.

The non-alkali-thickening acrylic resin 101 comprises a moisture permeable resin containing an alkali acid ester as a main component. Alternatively, the non-alkali-thickening acrylic resin 101 may comprise an acrylic acid ester mainly and a small amount of a urethane resin and/or a silicon resin.

As shown in FIG. 1, the epidemic prevention solidified matter 100 is provided with microcracks 104. Each of the microcracks 104 connects the lime hydrate particles 102 inside the solidified matter to each other and reaches to a surface of the epidemic prevention solidified matter 100. The microcracks 104 serve as exuding paths. A maximum width of the microcrack 104 is 0.01 to 100 μm, chiefly 1 to 10 μm.

The epidemic prevention solidified matter 100 further comprises fine titanium oxide particles 105. Since the titanium oxide particles 105 are comprised, the epidemic prevention solidified matter 100 becomes white and is improved in shielding property. Therefore, the base material 200 is shielded by the coating film-like epidemic prevention solidified matter 100, and, as a result, when a roof material is covered with the epidemic prevention solidified matter 100, it is possible to attain the light shielding and heat shielding effects against sunlight.

Since the titanium oxide particles 105 are comprised, wettability of the surface of the epidemic prevention solidified matter 100 is improved. Accordingly, a contact angle of a water droplet is reduced, and a moisture 106 thinly and widely adheres to the surface of the epidemic prevention solidified matter 100. Water such as rainwater, dew condensation water, and sprinkled water permeates through the microcracks 104 to dissolve the internal lime hydrate particles 102.

When the moisture 106 adheres to the surface of the epidemic prevention solidified matter 100, the moisture reaches to the internal lime hydrate particles 102 via the microcracks 104 to express alkalinity of pH 10 or more. The alkalinity lasts long and stably for a several months and inactivates pathogenic viruses such as an avian influenza virus and a porcine influenza virus on the surface of the epidemic prevention solidified matter 100.

Since the lime hydrate particles 102 which are dispersed and embedded in the form of solid particles shielded from the air inside the epidemic prevention solidified matter 100 and the surface of the epidemic prevention solidified matter 100 are communicated with each other via the microcracks 104, the internal lime hydrate particles 102 gradually exude out onto the solidified matter surface through the microcracks 104. Meanwhile, the lime hydrate particles 102 other than the exuded out lime hydrate particles 102, which are dispersed and embedded inside the solidified matter, are still in the state of being shielded from the air, they are not neutralized with a carbonate gas in the air nor are changed into calcium carbonate. Therefore, the antibacterial and antiviral effects are exhibited for a long time.

The moisture permeable resin shuts off water which is in the form of a liquid and allows passage of water vapor. Since the non-alkali-thickening resin 102 has the microcracks 104 and, further, since the resin 102 per se is the moisture permeable resin, the epidemic prevention solidified matter 100 is caused to be in a moist state, so that the lime hydrate particles 102 absorb outside water vapor. As a result, the lime hydrate particles 102 are dissolved in the moist environment without delay. Thus, the synergy effect that the alkaline component passes through the microcracks 104 to readily exude out onto the surface of the epidemic prevention solidified matter 100 is attained. Therefore, the epidemic prevention solidified matter 100 expresses the strong alkalinity of pH 10 or more even when the moisture 106 is in a small amount, i.e. even with the dew condensation water. Further, in the case where the moisture 106 is the rainwater or sprinkled water, the epidemic prevention solidified matter 100 of course expresses the alkalinity of pH 10 or more.

Hereinafter, a method for forming the epidemic prevention solidified matter 100 of the present embodiment will be described.

For a matrix, 15 to 75 parts by weight of an emulsion containing 5 to 45 parts by weight of a non-alkali-thickening resin, 10 to 50 parts by weight of lime hydrate particles which are in the form of a powder of particles of 50 μm or less, 7 to 10 parts by weight of titanium oxide which is in the form of a powder of particles of 10 μm or less, and 5 to 40 parts by weight of concentration adjustment water are used, and they are mixed and stirred to prepare a mixed slurry in an amount of 100 parts by weight as the entire mixed slurry. The secondary particles of the lime hydrate shown in FIG. 1 are dispersed inside the mixed slurry.

Next, the mixed slurry is coated on a surface of a formation object and is left to stand still until the mixed slurry is cured. The lime hydrate particles 102 are dispersed and embedded in the form of solid particles inside the solidified matter during the curing, and it is considered that the microcracks 104 extend from the secondary particles 103 of the lime hydrate particles 102 as shown in FIG. 1 during curing to be formed inside the acrylic resin 101. The factor for generation of the microcracks 104 is considered to be a shrinkage ratio difference between the acrylic resin 101 and the lime hydrate secondary particles 103 or to be a chemical reaction between the acrylic resin 101 and the lime hydrate secondary particles 103. The microcracks 104 are not easily recognized by human eyes, and it is considered that a width of each of the microcracks 104 is 0.01 to 100 μm.

When so required, a coloring agent, a spreadability enhancer, an antibacterial agent, an antiviral agent, a viscosity improver, a surfactant, a dispersant, an antifoaming agent, heat shielding particles, and the like may be contained in the mixed slurry as other auxiliary materials.

As the second embodiment, an epidemic prevention solidified matter according to the present invention may be prepared into a cake-like solid matter as a matrix preparation material by adding concentration adjustment water to an alkaline substance and an auxiliary material and kneading the mixture. The cake-like solid matter is transported to a site of formation and mixed and stirred with a non-alkali-thickening resin emulsion in the formation site to be prepared into a mixed slurry, thereby obtaining the matrix.

For example, 40 to 50 parts by weight of the cake-like solid matter and 50 to 60 parts by weight of the non-alkali-thickening resin emulsion may be used based on 100 parts by weight of the entire mixed slurry. As components of the cake-like solid matter, 10 to 25 parts by weight of the alkaline substance, 20 to 30 parts by weight of the concentration adjustment water, 1 to 3 parts by weight of a viscosity improver, and 1 to 3 parts by weight of a dispersant may be used.

By producing a large amount of the cake-like solid matter in a factory or the like in advance of formation and then transporting it to a site of the formation as required, it is possible to omit a step of mixing and stirring the alkaline substance such as the lime hydrate in the formation site (the mixing and stirring can cause scattering of lime hydrate and the like in the formation site to deteriorate the work environment), to improve workability in the formation site, and to prevent deterioration of the work environment in the formation site.

As the third embodiment, an epidemic prevention solidified matter according to the present invention is a coating film-like solidified matter which is formed on a surface of each of grains by coating, drying, and solidifying the mixed slurry on the surface of each of the grains such as pebbles, gravel, sand, pumice, cobbles, wood chip, metal grains, concrete grains, ceramic grains, and plastic grains. It is possible to readily attain the epidemic prevention by spreading or stacking the grains in a site of an epidemic prevention (road, passage, premise, etc.).

As a method of coating the mixed slurry on the surfaces of grains such as pebbles, a method of attaining the coating at once in which a large amount of the grains is immersed via a basket or the like into a container containing the mixed slurry, a method of coating by continuously spraying the mixed slurry from above onto the grains during conveyance on a conveyer, and the like are contemplated.

As the fourth embodiment, an epidemic prevention solidified matter according to the present invention is a coating film-like solidified matter which is formed on a surface of a sheet material by coating, dying, and solidifying the mixed slurry on the surface of the sheet material such as nonwoven cloth, cloth, paper, a mat, a carpet, a rug, an artificial grass, and a film. It is possible to readily attain the epidemic prevention by carpeting or attaching the sheet material in a site of an epidemic prevention (indoor wall, floor, ceiling, road, passage, premise, etc.).

As a method of coating the mixed slurry on the surface of the sheet such as nonwoven cloth, a method of spray-coating the mixed slurry using a coater, a method of coating the mixed slurry with a brush or roller, a method of printing the mixed slurry by using a printer, or the like may be employed.

### EXAMPLES

The inventors conducted evaluation tests in order to confirm effects of the epidemic prevention solidified matters of the present invention. As shown in Table 1, mixed slurries having compositions shown as Example 1 to Example 13 were prepared, and each of the prepared mixed slurries was coated on a slate material of a roof to form a solidified matter in the form of a coating film, followed by evaluation of 5 items, namely, high alkalinity exhibition, high alkalinity sustention, a heat shielding property, wettability, and workability. Also, in the case where the high alkalinity exhibition is excellent (⊚) and 3 or more items out of the 5 items are excellent (⊚), comprehensive evaluation of excellent (⊚) is given. In the case where the high alkalinity exhibition is excellent (⊚) and less than 3 items out of the 5 items are excellent (⊚), comprehensive evaluation of satisfactory (o) is given. In the case where the high alkalinity exhibition is poor (x), comprehensive evaluation of poor (x) is given.

TABLE 1

| | Matrix (mixed slurry) | | | | | | | | |
|---|---|---|---|---|---|---|---|---|---|
| | Main material | | | | Auxiliary material | | | | |
| | | | | Concentration | | | | Heat | |
| | Resin | Lime | Sodium | adjustment | Titanium | Viscosity | | shielding | |
| Sample | emulsion | hydrate | silicate | water | oxide | improver | Dispersant | particles | Total |
| Comp. | 50 | — | — | 25 | 10 | 10 | 5 | — | 100 |
| Ex. 1 | | | | | | | | | |
| Ex. 1 | 15 | 10 | — | 35 | 15 | 7 | 3 | 15 | 100 |
| Ex. 2 | 20 | 30 | — | 40 | 10 | — | — | — | 100 |
| Ex. 3 | 30 | 17 | — | 30 | 7 | 4 | 2 | 10 | 100 |
| Ex. 4 | 30 | 25 | — | 25 | 7 | 4 | 2 | 7 | 100 |
| Ex. 5 | 33 | 30 | — | 25 | 7 | 3 | 2 | — | 100 |
| Ex. 6 | 35 | 30 | — | 20 | 10 | 5 | — | — | 100 |
| Ex. 7 | 35 | 35 | — | 25 | 5 | — | — | — | 100 |
| Ex. 8 | 35 | 20 | 30 | 10 | 3 | 2 | — | — | 100 |
| Ex. 9 | 40 | 35 | — | 15 | 10 | — | — | — | 100 |
| Ex. 10 | 50 | — | 35 | 10 | 5 | — | — | — | 100 |
| Ex. 11 | 50 | 20 | — | 20 | 4 | 3 | 3 | — | 100 |
| Ex. 12 | 50 | 20 | — | 20 | 5 | 3 | 2 | — | 100 |
| Ex. 13 | 60 | 20 | — | 10 | 6 | 2 | 2 | — | 100 |
| Ex. 14 | 50 | 20 | — | 20 | 5 | — | 5 | — | 100 |
| Ex. 15 | 75 | 20 | — | 5 | — | — | — | — | 100 |

| | Evaluation | | | | | |
|---|---|---|---|---|---|---|
| | High | High | Heat | | | |
| | alkalinity | alkalnity | shielding | | | Comprehensive |
| Sample | exhibition | sustention | property | Wettability | Workability | evaluation |
| Comp. | X | X | ◯ | ◯ | ⊚ | X |
| Ex. 1 | | | | | | |
| Ex. 1 | ◯ | ◯ | ⊚ | ◯ | ⊚ | ◯ |
| Ex. 2 | ⊚ | ⊚ | ◯ | ⊚ | Δ | ⊚ |
| Ex. 3 | ⊚ | ◯ | ⊚ | ⊚ | ⊚ | ⊚ |
| Ex. 4 | ⊚ | ⊚ | ⊚ | ⊚ | ⊚ | ⊚ |
| Ex. 5 | ⊚ | ⊚ | ◯ | ⊚ | ⊚ | ⊚ |
| Ex. 6 | ⊚ | ⊚ | ◯ | ◯ | ◯ | ◯ |
| Ex. 7 | ⊚ | ⊚ | ◯ | ◯ | Δ | ◯ |
| Ex. 8 | ⊚ | ⊚ | ◯ | ◯ | Δ | ◯ |
| Ex. 9 | ⊚ | ⊚ | ◯ | ⊚ | Δ | ⊚ |
| Ex. 10 | ⊚ | ⊚ | ◯ | ◯ | Δ | ◯ |
| Ex. 11 | ⊚ | ◯ | ◯ | ◯ | ⊚ | ◯ |
| Ex. 12 | ⊚ | ⊚ | ◯ | ◯ | ⊚ | ⊚ |
| Ex. 13 | ◯ | ⊚ | ◯ | ⊚ | ◯ | ◯ |
| Ex. 14 | ⊚ | ◯ | ⊚ | ◯ | ⊚ | ⊚ |
| Ex. 15 | ⊚ | ◯ | Δ | ◯ | Δ | ◯ |

In Table 1, a commercially available coating (aqueous acrylic resin) was used as Comparative Example 1. In Example 1 to Example 13, an acrylic acid ester copolymer aqueous emulsion (“K-6200S” manufactured by Shin-Nakamura Chemical Co., Ltd.) was used as the resin emulsion, and dolomite-based lime hydrate (“White D” manufactured by Tagen Lime Industry) was used as lime hydrate. As the resin emulsion of Example 10, 3 parts by weight of a silicon resin emulsion was added to 47 parts by weight of the acrylic acid ester copolymer aqueous emulsion. As the resin emulsion of Example 14, 10 parts by weight of a urethane resin emulsion was added to 40 parts by weight of the acrylic acid ester copolymer aqueous emulsion. Also, rutile type titanium oxide was used as titanium oxide; “Viscon A-8” manufactured by Shin-Nakamura Chemical Co., Ltd. was used as a viscosity improver; and “Poiz 521” manufactured by Kao Corporation was used as a dispersant.

Referring to Table 1, each of Comparative 1 and Examples 1, 3 to 13, and 15 was formed on a commercially available iron plate (20×20 cm, thickness: 3 mm) and then was subjected to an outdoor exposure test to measure pH of a surface by using a commercially available pH test paper. The pH measurement was conducted by placing a sheet of the pH test paper on the surface which was wet with water and, 1 to 30 seconds later, comparing a color of the sheet of pH test paper with the comparative color table to determine a pH value. When the pH value at the start of the outdoor exposure test was 12 or more, the high alkalinity exhibition was evaluated as excellent (⊚). When the pH value was less than 12, the high alkalinity exhibition was evaluated as satisfactory (o), not satisfactory (Δ), or poor (x) in accordance with the value. When a period during which pH is 10 or more is over 6 months, the high alkalinity sustention was evaluated as excellent (⊚). When the period is less than 6 months, the high alkalinity sustention was evaluated as satisfactory (o), not satisfactory (Δ), or poor (x) in accordance with the value.

Referring to Table 1, Example 2 was formed on commercially available pebbles (average grain diameter: about 2 cm) and then was subjected to the outdoor exposure test to measure pH on a surface by using a commercially available pH test paper. Measurement of pH and evaluation criteria were the same as those described above.

Referring to Table 1, Example 14 was formed on a material obtained by attaching commercially available nonwoven cloth (nonwoven cloth wiper purchased from MonotaRO, size: 250×250 mm) to a veneer board and then was subjected to the outdoor exposure test to measure pH on a surface by using a commercially available pH test paper. Measurement of pH and evaluation criteria were the same as those described above.

Referring to Table 1, each of Examples 12 and 13 was prepared into a cake-like solid mass by kneading the components other than the resin emulsion into a mortar and then storing in a tightly-sealed polyethylene bag at room temperature for one week. After that, the solid mass was mixed with the resin emulsion with stirring before the coating to prepare a slurry to be coated.

As shown in the evaluations of Table 1, Comparative Example 1 cannot express the high alkalinity of pH 10.0 or more to be evaluated as poor in the comprehensive evaluation. Among Examples 1 to 15, each of Example 2 to 5, 9, 12, and 14 expresses the high alkalinity of pH 10.0 or more and is evaluated as excellent (⊚) in terms of 3 evaluation items out of the 5 evaluation items to be evaluated as excellent (⊚) in the comprehensive evaluation. Each of Examples 1, 6 to 8, 10 to 11, 13, and 15 expresses the high alkalinity of 10.0 or more but is evaluated as excellent (⊚) in terms of less than 3 evaluation items out of the 5 evaluation items to be evaluated as satisfactory (o) in the comprehensive evaluation.

As the lime hydrate, the one used as a fertilizer was used, but industrial lime hydrate is also usable. Any lime hydrate is usable insofar as it is called an environment improvement and adjustment material and is categorized into sundries. Of course, when calcium hydroxide having high purity is required for a special usage, calcium hydroxide of a reagent grade may be used. Also, as titanium oxide, either one of anatase type titanium oxide and rutile type titanium oxide may be used.

Further, the inventors conducted an evaluation test to confirm an effect of preventing deposition and invasion of pest of the preventive method of the present invention by using each of the matrixes of Example 4, Example 5, and Example 13 (see Example 16 to Example 18 described below).

### Example 16

Anoplophora malasiaca developed to the adult stage makes a hole in a tree around June and lays eggs in the hole. Since hatched larvae eat away the internal part of the tree, the tree can die down if it is young though the tree can survive if it has grown to a certain size. As a conventional prevention method, a material obtained by dissolving lime hydrate into water, followed by mixing with lard, has been pasted on trees. However, since the material is undesirably washed away when it rains, it has been necessary to repeat the work.

Accordingly, the inventors formed a solidified matter by using the matrix of Example 4 in Table 1 and coating the matrix on 7 Chinese lemon trees in a farm in April in such a manner that the matrix was coated up to a height of about 30 cm from the bottom of each of the trees. Other Chinese lemon trees in the farm were not coated with the matrix to be used as controls. As a result of investigation in May, some cracks were confirmed on a surface where the solidified matter was formed, but the solidified matter was not washed away, and the leaves were not blasted. However, some of sprouts on which the matrix was coated were blasted. Upon report of breeding of longicorn in the middle of June, the investigation was conducted in July to confirm that encroachment of the Chinese lemon trees by the longicorn in the experiment region was minor as compared to the control region. In the investigation in December, too, it was confirmed that the state of the solidified matter formation surface was not changed from that of the investigation in May and that the Chinese lemon trees were not harmed at all.

### Example 17

Dermanyssus gallinae which is a kind of nocturnal hematophagous ticks attacks chickens particularly violently and has a habit of living in a narrow space between structures inside a poultry house. The chicken attacked by Dermanyssus gallinae suffers from generalized itchiness, and the itchiness can cause a reduction in egg production in the case where the chicken is a layer hen or can result in death of the chicken.

Accordingly, the inventors used the matrix of Example 5 in Table 1 on Dermanyssus gallinae by coating the matrix on several contact portions between a metal feeding trough and braces which are the habitat of Dermanyssus gallinae in a chicken layer house with a brush in the spring, and other portions which were not coated with the matrix were used as controls. As a result, the habitat of Dermanyssus gallinae was not confirmed at all on the portions where the matrix was coated in the experiment region even in the summer when breeding of Dermanyssus gallinae is generally most active, while the habitat and deposition similar to those happened in the past were confirmed in the control region. Further, it was confirmed that the Dermanyssus gallinae habitat and deposition prevention effect in the experiment region was perfect throughout the year.

### Example 18

A support rod used for constructing a cultivation area of fish, oyster, or seaweed is a long plastic rod and is thrust into the sea bottom. When the support rod is used for a long time, a large amount of shell is deposited on a portion of the rod under the sea, and, therefore, the rod not only becomes a haven for pathogenic microbes and microscopic plants and animals but also becomes a heavy load when pulling up the rod for removing or repairing the cultivation area to make the work considerably difficult.

Accordingly, the inventors used rods on each of which a solidified coating film was formed by coating and drying the matrix of Example 13 of Table 1 after coating and drying a primer called Mitchakulon and started an experiment in the fall by thrusting 5 support rod on which the solidified coating film was formed and 5 ordinary support rods in a control region into the sea bottom in a seaweed farm in the Ariake sea. As a result of investigation of the rods after about one year, shell deposition was confirmed in the control region, while the shell deposition was not confirmed in the experiment region. A weight of the shell deposition in the experiment region after about 2 years was about 5% of that of the control region.

### INDUSTRIAL APPLICABILITY

The epidemic prevention solidified matter according to the present invention is widely usable for epidemic prevent in livestock barns, poultry houses, farms, general buildings, passages, roads, rivers, parks, cultivation area, and the like.

### REFERENCE SINGS LIST

100: epidemic prevention solidified matter
101: non-alkali-thickening acrylic resin
102: lime hydrate particles
103: lime hydrate fine particles
104: microcracks (exuding paths)
105: titanium oxide particles
106: moisture
200: base material

1. An epidemic prevention method comprising:
providing a mixed slurry comprising an emulsion of a non-alkaline-thickening resin which is free from a coagulation-attributable viscosity increase even when mixed with an alkaline substance, an alkaline substance of which a diluted solution has pH 10.0 or more, and concentration adjustment water, the mixed slurry being obtainable by mixing and stirring the emulsion, the alkaline substance, and the concentration adjustment water;
coating, drying, and solidifying the mixed slurry on a surface of an object to form on the surface of the object a solidified matter in which the alkaline substance is internally dispersed and embedded in the form of solid particles and microcracks provide communication between the solid particles of the internal alkaline substance and an outer surface;
allowing a moisture adhered to a surface of the solidified matter to permeate through the microcracks thereby to dissolve the solid particles of the internal alkaline substance; and
inactivating a microbe or a virus on the surface of the solidified matter by exhibiting pH 10.0 or more on the surface of the solidified matter.

2. The epidemic prevention method according to claim 1, wherein:
the alkaline substance comprises at least one or a mixture of plural kinds selected from oxides of quicklime and magnesium oxide, lime hydrate, magnesium hydroxide, hydroxides of sodium hydroxide and potassium hydroxide, and alkali salts of sodium carbonate, potassium carbonate, sodium silicate, potassium silicate, sodium phosphate, potassium phosphate, sodium acetate, and potassium acetate.

3. The epidemic prevention method according to claim 1, wherein:
the mixed slurry comprises 15 to 75 parts by weight of the emulsion containing 5 to 45 parts by weight of the non-alkali-thickening resin, 10 to 50 parts by weight of the alkaline substance, and 5 to 40 parts by weight of the concentration adjustment water based on 100 parts by weight of the entire mixed slurry.

4. The epidemic prevention method according to claim 1, wherein:
the non-alkali-thickening resin is at least one resin or a resin mixture of plural kinds of resins selected from an acrylic resin, a urethane resin, a fluorine resin, a silicon resin, a polyester resin, a polyethylene resin, a polystyrene resin, an epoxy resin, and a polyphenol resin.

5. The epidemic prevention method according to claim 1, wherein:
the mixed slurry further includes one or more of a coloring agent, a spreadability enhancer, an antibacterial agent, an antiviral agent, a viscosity improver, a surfactant, a dispersant, an antifoaming agent, and heat shielding particles as an auxiliary agent.

6. The epidemic prevention method according to claim 1, wherein:
the mixed slurry further includes heat shielding particles selected from titanium oxide, perlite, ferrite, and ceramic particles as an auxiliary agent.

7. The epidemic prevention method according to claim 1, wherein:
the object is a grain selected from pebbles, gravel, sand, pumice, cobbles, wood chip, metal grains, concrete grains, ceramic grains, and plastic grains.

8. The epidemic prevention method according to claim 1, wherein:
the object is a sheet material selected from nonwoven cloth, cloth, paper, a mat, a carpet, a rug, an artificial grass, and a film.

9. An epidemic prevention method, comprising:
providing a mixed slurry comprising an emulsion of a non-alkaline-thickening resin which is free from a coagulation-attributable viscosity increase even when mixed with an alkaline substance, an alkaline substance of which a diluted solution has pH 10.0 or more, and concentration adjustment water, the mixed slurry being obtainable by mixing and stirring the emulsion, the alkaline substance, and the concentration adjustment water; and
casting the mixed slurry into a molding die, followed by drying and solidification, to obtain the solidified matter in the form of a block or a flat plate in which the alkaline substance is internally dispersed and embedded in the form of solid particles and the microcracks provide communication between the solid particles of the internal alkaline substance and an exterior surface.

10. The epidemic prevention method according to claim 1, further comprising the step of:
subjecting an exterior of the solidified matter to cleaning by brushing or cleaning with high pressure water.

11. The epidemic prevention method according to claim 1, where said step of allowing a moisture adhered to a surface of the solidified matter to permeate through the microcracks causes a pH 10.0 or more to be exhibited on the surface of the solidified matter thereby to prevent a pest, a small animal, or a microscopic animal from depositing on the surface of the solidified matter and invading beyond a region where the solidified matter is formed.

12. The epidemic prevention method according to claim 11,
wherein the object is selected from a trunk or branches of a tree which is subject to encroachment by insects, microscopic animals and plants, microbes, a timber of the tree, a structure inside a livestock barn or a house which can be a habitat of microscopic animals and plants, microbes, a structure in water on which small animals or microscopic animals and plants can be deposited, and a soil or a ground to form the solidified matter in the form of a coating film on the surface of the object;
allowing a moisture adhered to a surface of the solidified matter to permeate through the microcracks thereby to dissolve the solid particles of the internal alkaline substance; and
causing pH 10.0 or more to be exhibited on the surface of the solidified matter thereby to prevent a insect, a small animal, a microscopic animal, microscopic plants, fungus or mold from depositing on the surface of the solidified matter and invading beyond a region where the solidified matter is formed.

13. A method for preventing epidemic among livestock, the method comprising:
(i) applying a composition to a substrate in proximity to livestock, where the composition is formed by combining (a) an emulsion of a non-alkaline-thickening resin which is free from a coagulation-attributable viscosity increase even when mixed with an alkaline substance, (b) an alkaline substance of which a diluted solution has a pH of 10.0 or more, and (c) water; and
(ii) allowing the composition to solidify and thereby form a coating on the substrate, where the coating includes domains of the alkaline substance dispersed within the resin and where the coating is characterized by including microcracks that provide communication between the domains of the alkaline substance and the outer surface of the coating, whereby moisture permeates the microcracks and interacts with the domains of alkaline substance and thereby causes the outer surface of the coating to exhibit the pH of 10.0 or more, thereby inhibiting epidemic among livestock.
